(19) United States
(12) Patent Application Publication
(10) Pub. No.: US 2026/0183098 A1
(43) Pub. Date: Jul. 2, 2026

(54) ARTIFICIAL SUPPORT FOR BREAST RECONSTRUCTION

(71) Applicant: T&R BIOFAB CO., LTD., Siheung-si (KR)

(72) Inventors: In Ho KIM, Seongnam-si (KR); Song Ho KIM, Seongnam-si (KR); Chae Hyeon RYU, Seongnam-si (KR)

(21) Appl. No.: 18/856,870

(22) PCT Filed: Apr. 18, 2022
(86) PCT No.: PCT/KR2022/005529

§ 371 Date: Oct. 14, 2024

(30) Foreign Application Priority Data
Apr. 15, 2022 (KR) 10-2022-0047123

(51) Int. Cl. A61F 2/00; A61F 2/12; A61L 27/24; A61L 27/36

(52) U.S. Cl. CPC A61F 2/0063; A61F 2/12; A61L 27/24; A61L 27/3633; A61F 2220/0008; A61F 2220/0075

(57) ABSTRACT

The disclosed invention relates to an artificial support in the form of a sheet surrounding a breast implant and, in one example, the artificial support includes: a support portion having a plurality of divided surfaces radially formed surrounding the curved surface of the breast implant; and a frame portion which is coupled to the support portion and has a plurality of branched frames extending radially along the divided surfaces.

### TECHNICAL FIELD

The present disclosure relates to an artificial support surrounding a breast implant inserted into a human body during breast reconstruction.

### BACKGROUND

Breast reconstruction is a plastic surgery which restores the original breast shape to a female patient who has undergone mastectomy for a breast cancer treatment. According to the Korean Breast Cancer Society, in recent years, breast cancer has been reported to occur in 13.5% of 30's women and 38.1% of 40's women in Korea. In addition, it is reported that most women suffer from serious psychological distress after breast cancer treatment, as their quality of life is reduced due to mastectomy that occurs due to breast cancer.

Breast reconstruction which is performed to improve the quality of life of these women is performed using autologous tissue flaps and silicone breast implants after determining oncological safety. Autologous tissue flap surgery has the disadvantages of additional scarring at the donor site, asymmetry between the left and right breasts, and a long recovery period after surgery. Therefore, recently, there is a trend toward performing more silicone breast implants which are simple and have fewer side effects such as muscle damage. In addition, in the silicone artificial breast implantation process, in order to reduce postoperative side effects and increase plastic surgery satisfaction, an acellular dermal matrix (ADM) is used as support to surround the implant or fix and suture it to the human body after inserting the silicone artificial breast.

Acellular allogeneic dermis is a medical material for dermal regeneration produced through a decellularization process using dermal tissues obtained from the skin of a donated cadaver and a medical material which may be applied to treat various skin damages. Acellular allogeneic dermis has the disadvantages of being considerably more expensive than other medical materials, limited in supply, and difficult to maintain consistent quality. Therefore, a new technique which may replace the acellular allograft used in breast reconstruction is needed, and for this purpose, natural polymer or synthetic polymer materials with excellent biocompatibility are gradually being applied.

On the other hand, artificial supports used for reducing the side effects of silicone artificial breast insertion and to fix and suture them to the human body are currently provided in a simple sheet form without a specific shape. Therefore, at the surgical site, an artificial support sheet is cut arbitrarily and attached to surround the breast implant before being inserted into the human body. In addition, since the cut is roughly made to fit the eye, there are portions in which the artificial support does not properly cover the breast implant or portions in which they overlap excessively and becomes thick. In addition, at the time of cutting an artificial support, it is quite difficult to determine a shape thereof while considering the strength of the point in which it is fixed and sutured to the human body which can ultimately have a negative impact on the success rate of breast reconstruction.

### SUMMARY

The present disclosure is for the purpose of providing an artificial support which may be effectively fixed to a breast implant and stably bonded to a body while reducing side effects at the time of being inserted into the body.

Here, the technical matters that the present disclosure seeks to solve are not limited to the matters described above and other matters which are not described will be clearly understood by those skilled in the art from the description of the invention which will be described below.

The present disclosure relates to an artificial support in the form of a sheet surrounding a breast implant and, in one example, the artificial support includes: a support portion having a plurality of divided surfaces radially formed surrounding the curved surface of the breast implant; and a frame portion which is coupled to the support portion and has a plurality of branched frames extending radially along the divided surfaces.

According to an example embodiment, the frame portion may be concentric with the support portion.

Also, it is preferable that a surface area of the support portion be larger than a surface area of the frame portion and a strength of the frame portion be greater than a strength of the support portion.

On the other hand, the branched frames may include terminal portions protruding from the divided surfaces.

Furthermore, the frame portion, at the time of being aligned concentrically with respect to the breast implant, may have a diameter so that the terminal portions of the branched frames extend beyond a front surface of the breast implant to a rear surface.

In addition, the terminal portions of the branched frames may include suture points.

Thus, the terminal portion of each branched frame may be surround such that it extends beyond the front surface of the breast implant to the back surface in a state in which the breast implant is aligned concentrically with respect to the frame portion and a suture thread passes through the suture point of the terminal portion placed on the back surface of the breast implant and is ligated so that the artificial support is fixed to the breast implant.

Moreover, according to an example embodiment, the support portion and the frame portion may be structures produced through a 3D printer using a thermoplastic synthetic polymer as a material.

Also, the artificial support of the present disclosure may further include a biocompatible base portion surrounding the support portion and the frame portion.

In one example, the base portion may be a natural polymer selected from the group consisting of collagen and tissue-derived decellularized extracellular matrix (dECM).

Furthermore, the base portion may be formed by applying a gel-type natural polymer to the surface or an inside and a surface of the support portion and the frame portion and then freeze-drying it.

It is preferable that the base portion surround the support portion and the frame portion so that they are not exposed to an outside.

The artificial support of the present disclosure having the above configuration has a support portion having a plurality of divided surfaces surrounding the breast implant and the frame portion structurally supports this so that the two-dimensional sheet-shaped artificial support may flexibly surround the curved breast implant.

In addition, the artificial support may be sutured using the terminal portion of the branched frame of the frame portion, thereby making it possible to firmly and easily connect the artificial support to the breast implant.

In addition, the artificial support of the present disclosure may significantly reduce the risk of inflammation induction and further promote cell proliferation by further providing a base portion with excellent biocompatibility.

Here, the technical effects which may be obtained through the present disclosure are not limited to the effects described above and other effects which are not described may be clearly understood by those skilled in the art from the description of the invention which will be described below.

### BRIEF DESCRIPTION OF THE DRAWINGS

The following drawings attached to this specification illustrate preferred embodiments of the present disclosure and, together with the detailed description of the invention which will be described below, serve to further understand the technical idea of the present disclosure;

therefore, the present disclosure need not to be construed as being limited to matters described in such drawings.

FIG. 1 is a diagram showing an artificial support according to an example embodiment of the present disclosure.

FIG. 2 is a diagram showing a modified example of the artificial support of FIG. 1.

FIG. 3 is a diagram showing a front view of the artificial support of FIG. 2 attached to the breast implant.

FIG. 4 is a diagram showing a back surface of the artificial support of FIG. 2 attached to the breast implant.

FIG. 5 is a diagram showing a state of a suture being tied to the artificial support of FIG. 4.

FIG. 6 is a diagram showing an artificial support according to another example embodiment of the present disclosure.

EXPLANATION OF REFERENCE NUMERALS

| | |
|---|---|
| 100: Artificial support | 110: Support portion |
| 112: Divided surface | 120: Frame portion |
| 122: Branched frame | 124: Terminal portion |
| 126: Suture point | 128: Suture thread |
| 130: Base portion | 200: Breast implant |

### DETAILED DESCRIPTION

The present disclosure may have various modifications and embodiments and specific embodiments will be described in detail below.

Here, this is not intended to limit the present disclosure to a specific embodiment, but needs to be understood to include all modifications, equivalents or substitutes included in the spirit and technical scope of the present disclosure.

In the present disclosure, it needs to be understood that terms such as “comprises” or “have” are intended to specify the presence of a feature, number, step, operation, constituent element, part, or combination thereof described in the specification, but do not exclude in advance the possibility of the presence or addition of one or more other features, numbers, steps, operations, components, parts, or combinations thereof.

Also, in the present disclosure, if a part such as a layer, membrane, region, or plate is described as being “on” another part, this includes not only cases in which one part is “directly above” another part, but also cases in which there is another part in between. On the other hand, when a part such as a layer, membrane, region, or plate is described as being “below” another part, this includes not only cases in which it is “directly below” the other part, but also cases in which there are other parts in between. Furthermore, in the present application, the term “placed on” may include a case in which it is placed on the lower side as well as the upper side.

The present disclosure relates to an artificial support 100 in the form of a sheet surrounding a breast implant 200. The artificial support 100 is a structure which induces the breast implant 200 that is a type of xenobiotic substance inserted into the human body to stably bind to body tissues without side effects. The artificial support 100 surrounds the surface of the breast implant 200 to support it and promote cell adhesion and cell and tissue growth, thereby enabling it to be combined with body tissues. For example, acellular allograft (ADM) has been used as an artificial support in the past.

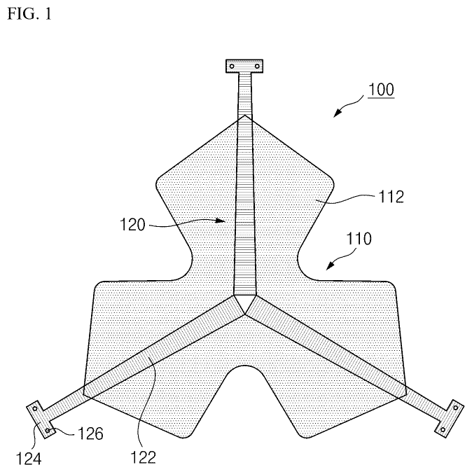

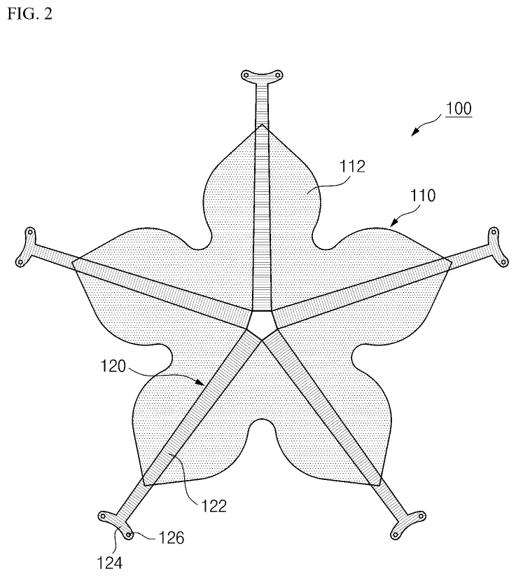

FIG. 1 and FIG. 2 show the artificial support 100 according to an example embodiment of the present disclosure. Referring to the attached drawings, the artificial support 100 of the present disclosure includes a support portion 110 and a frame portion 120.

The support portion 110 is a portion with which the surface of the breast implant 200 is mainly covered. In addition, in the support portion 110, a plurality of divided surfaces 112 are formed radially so that the support portion 110 in the form of a flat sheet may flexibly surround the curved surface of the breast implant 200. That is to say, except for the central part in which the divided surfaces 112 are radially connected to each other, the divided surfaces 112 are spread out from each other to form a shape like a corolla. In addition, due to this opened shape, the support portion 110 appropriately surrounds the curved surface of the breast implant 200 without major wrinkling.

There is no specific limitation on the number of divided surfaces 112 and they are designed in an appropriate number which may be well bonded to the surface of the breast implant 200. FIG. 1 shows a case in which the number of divided surfaces 112 is 3 as an example and FIG. 2 shows a case in which the number of divided surfaces 112 is 5 as an example.

The frame portion 120 is a portion which is combined with the support portion 110 to form an integral portion. In addition, in the frame portion 120, a plurality of branched frames 122 are formed radially along the divided surfaces 112 of the support portion 110. That is to say, each of the divided surfaces 112 includes one branched frame 122, and thus the branched frames 122 serve as a kind of skeleton for the divided surfaces 112. In the shown example embodiment, the frame portion 120 and the support portion 110 are concentric with each other, thereby forming an overall balanced structure of rotational symmetry.

Here, referring to the drawings, comparing the configuration of the support portion 110 and the frame portion 120 according to an example embodiment of the present disclosure, the surface area of the support portion 110 may be larger than the surface area of the frame portion 120. On the other hand, the strength of the frame portion 120 may be formed to be larger than the strength of the support portion 110.

This is in consideration of the main functions of the support portion 110 and the frame portion 120, respectively. This is because the support portion 110 is designed to secure a region in which cells may grow and the frame portion 120 is designed to support the load of the artificial support 100 and the breast implant 200. Note that this is only a distinction based on the main functions of the support portion 110 and the frame portion 120. In addition, for example, since the frame portion 120 also has the effect of cell growth, in this respect, the frame portion 120 needs to be viewed as including some of the functions of the support portion 110.

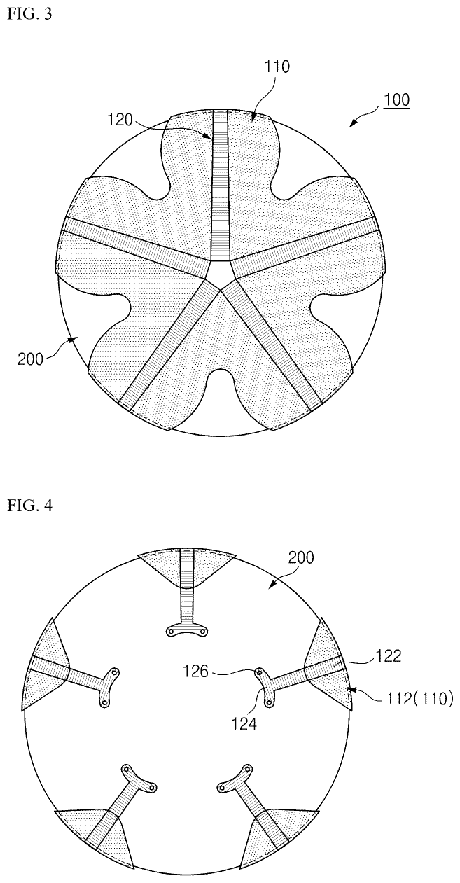

The support portion 110 may cover a certain percentage or more of the surface area of the breast implant 200, for example, 60% or more of the front surface of the breast implant 200 so that the support portion 110 secures a sufficient area on which cells may grow. FIG. 3 is a diagram showing a front side of the artificial support 100 of FIG. 2 combined with the breast implant 200 in a state in which the support portion 110 covers a wide area of the front side of the breast implant 200.

The frame portion 120 which is combined with the support portion 110 to form an integral body serves to secure durability against the load of the breast implant 200 by forming the skeleton of the artificial support 100 like the ribs of an umbrella. For this purpose, it is preferable that the frame portion 120 have a stronger strength than the support portion 110. The strength of the frame portion 120 may be achieved in a variety of ways such as by varying the material, thickness, or by constructing the frame portion 120 with a composite material.

For reference, the relative comparison of the surface area and the strength of the support portion 110 and the frame portion 120 is based on a preferred example embodiment. According to a modified example embodiment, the surface area of the frame portion 120 may be made equal to or larger than that of the support portion 110 so that the frame portion 120 has sufficient strength.

On the other hand, according to an example embodiment of the present disclosure, the branched frame 122 may include a terminal portion 124 which protrudes from the divided surface 112 of the support portion 110, more specifically protrudes from the distalmost end of the divided surface 112.

Furthermore, the terminal portion 124 of the branched frame 122 may have a diameter extending beyond the front surface of the breast implant 200 to the rear surface when the artificial support 100 is aligned concentrically with respect to the breast implant 200.

Thus, the artificial support 100 of the present disclosure may surround the terminal portion 124 of each branch frame beyond the front side to a part of the back surface of the breast implant 200 while the breast implant 200 is aligned concentrically with respect to the frame portion 120. FIG. 4 is a diagram showing the back surface of the artificial support 100 of the present disclosure at the time of being combined with the breast implant 200, and as shown, the terminal portion 124 of the branched frame 122 extends beyond the front surface of the breast implant 200 to the rear surface.

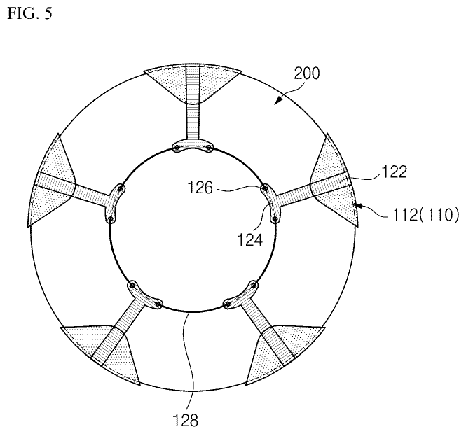

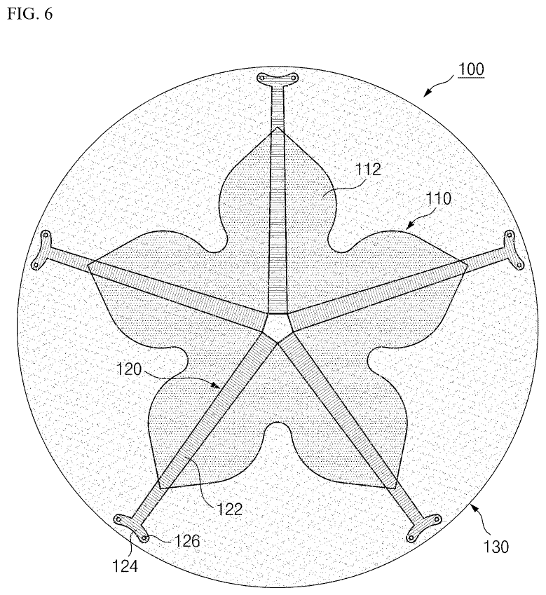

In addition, the terminal portion 124 of the branched frames 122 includes a suture point 126. By passing a suture thread 128 through the suture point 126 of the terminal portion 124 located at the rear side of the breast implant 200 and ligating it, the artificial support 100 may be stably fixed to the breast implant 200. FIG. 5 shows a state in which the artificial support 100 of FIG. 4 is stably connected to the breast implant 200 by fastening the suture thread 128.

Here, the suture point 126 means a point through which the suture thread 128 may pass and has a configuration of a through hole as shown. In addition, the suture point 126 needs to be understood as having a broad concept which includes all cases in which the material or the gap structure of the terminal portion 124 of the branched frames 122 allows the penetration of the suture thread 128, thereby forming the suture point 126 even if the suture point 126 is provided or a separate through hole is not formed.

The terminal portions 124 of the branched frames 122 which extend radially, converge again toward the center at the time of being folded toward the rear of the breast frame. Thus, the artificial support 100 may be firmly fixed to the breast implant 200 by ligating the suture thread 128 using the suture point 126 of the terminal portion 124. Therefore, in the example embodiment of FIG. 5, not only does the frame portion 120 have a strength which may withstand the load of the breast implant 200, but particularly, the terminal portion 124 of the branched frame 122 needs to have a strength (for example, various strengths such as a tensile strength, a burst strength, and a suture strength) which does not tear or stretch even under the tensile force of the ligated suture thread 128.

In addition, the terminal portion 124 included in the suture point 126 may be formed into a curved shape in which it forms a trajectory similar to a circle or an ellipse when the branched frame 122 is folded toward the rear of the breast frame (refer to FIG. 5). Since the shape of the terminal portion 124 has a similar trajectory to the circle formed by the suture thread 128, an excessive force is not applied to the suture point 126 and the risk of the suture point 126 breaking is reduced accordingly.

On the other hand, the artificial support 100 of the present disclosure may be made of various materials, for example, acellular dermal membrane (ADM), but may also be made of a synthetic polymer which may eliminate various disadvantages of acellular dermal membrane. For example, the support portion 110 and the frame portion 120 may be structures produced using a 3D printer using a thermoplastic synthetic polymer as a material.

The thermoplastic synthetic polymer applicable to the present disclosure may be at least one thermoplastic synthetic polymer selected from the group consisting of caprolactone, dioxanone, glycolide, lactide, propylene, ethylene, trimethylene, vinylchloride, butadiene, methyl methacrylate, carbonate, acrylic acid, 2-hydroxyethyl methacrylate, and polyethylene terephthalate.

These thermoplastic synthetic polymers may form a structure using a 3D printer which extrudes molten material and the thermoplastic synthetic polymers may be provided in a granule type, a powder type, a filament type, or the like to suit the 3D printer to which they are applied.

A polymer structure formed through a material extrusion method has different mechanical properties in accordance with the line width, the thickness, the structure, the material, or the like of the extruded material and these mechanical properties may be adjusted in accordance with the purpose of use, surgical site, and required properties. Therefore, in the artificial support 100 of the present disclosure, different stiffnesses of the support portion 110 and the frame portion 120 may be implemented through the line width, the thickness, the material, the single pattern structure, the combination of two or more single patterns, and the like of the extruded thermoplastic synthetic polymer.

On the other hand, FIG. 6 is a diagram showing the artificial support 100 according to another example embodiment of the present disclosure and the artificial support 100 of FIG. 6 further includes a biocompatible base portion 130 surrounding the support portion 110 and the frame portion 120.

Here, the base portion 130 may be a natural polymer selected from the group consisting of collagen and tissue-derived decellularized extracellular matrix (dECM). The collagen group includes atelocollagen, telocollagen, and the like and the tissue-derived decellularized extracellular matrix (dECM) group may include all tissue-derived decellularized extracellular matrices such as porcine tissue-derived, bovine tissue-derived, porcine tendon-derived, and bovine tendon-derived.

The base portion 130 may be formed by applying a gel-type natural polymer to the surface or the inside and surface of the support portion 110 and the frame portion 120 and then freeze-drying it. A gel-type natural polymer is converted into a sponge-type polymer through a freeze-drying process and the converted natural polymer and synthetic polymer are physically combined.

The base portion 130 is provided to assist or promote more stable bonding to a living body when the artificial support 100 described above is inserted into the human body. That is to say, when an edge of the artificial support 100 made of a synthetic polymer material comes into direct contact with body tissues, there is a possibility of inflammation due to some sharp or pointed portions. Thus, the base portion 130 which has excellent biocompatibility prevents direct contact with the artificial support 100, thereby preventing inflammation during insertion into the body.

In addition, when the artificial support 100 made of a synthetic polymer material is inserted into the body, it is difficult for cells and tissues in the body to attach and grow on the surface of the synthetic polymer. Thus, the natural polymer combined with the support portion 110 and the frame portion 120 of the artificial support 100 acts as a base plate for cell and tissue growth.

In this way, by adding the base portion 130 of the natural polymer, cell proliferation may be promoted more than when the artificial support 100 of the synthetic polymer material is used. This is because the base portion 130 of the natural polymer material has excellent biocompatibility which is the ability to exist in harmony with tissues without causing harm to the body.

Therefore, it may be preferable for the base portion 130 to be formed to a sufficient size to surround the support portion 110 and the frame portion 120 so that they are not exposed to the outside.

The present disclosure has been described in more detail above through drawings and examples. Here, the configuration described in the drawings or the examples described in this specification is only an embodiment of the present disclosure and does not represent all of the technical ideas of the present disclosure. Thus, it should be understood that there may be various equivalents and modifications which may be substituted for these at the time of this application.

### INDUSTRIAL APPLICABILITY

The present disclosure is appropriate for use as an artificial support surrounding a breast implant inserted into a human body during breast reconstruction.

1. An artificial support in the form of a sheet surrounding a breast implant, the artificial support comprising:
a support portion having a plurality of divided surfaces radially formed surrounding the curved surface of the breast implant; and
a frame portion which is coupled to the support portion and has a plurality of branched frames extending radially along the divided surfaces.

2. The artificial support of claim 1, wherein the frame portion is concentric with the support portion.

3. The artificial support of claim 1, wherein a surface area of the support portion is larger than a surface area of the frame portion, and
a strength of the frame portion is greater than a strength of the support portion.

4. The artificial support of claim 3, wherein the branched frames include terminal portions protruding from the divided surfaces.

5. The artificial support of claim 4, wherein the frame portion,
at the time of being aligned concentrically with respect to the breast implant, has a diameter so that the terminal portions of the branched frames extend beyond a front surface of the breast implant to a rear surface.

6. The artificial support of claim 5, wherein the terminal portions of the branched frames include suture points.

7. The artificial support of claim 6, wherein the terminal portion of each branch frame is surround such that it extends beyond the front surface of the breast implant to the back surface in a state in which the breast implant is aligned concentrically with respect to the frame portion and a suture thread passes through the suture point of the terminal portion placed on the back surface of the breast implant and is ligated so that the artificial support is fixed to the breast implant.

8. The artificial support of claim 1, wherein the support portion and the frame portion are structures produced through a 3D printer using a thermoplastic synthetic polymer as a material.

9. The artificial support of claim 8, further comprising:
a biocompatible base portion surrounding the support portion and the frame portion.

10. The artificial support of claim 9, wherein the base portion
is a natural polymer selected from the group consisting of collagen and tissue-derived decellularized extracellular matrix (dECM).

11. The artificial support of claim 10, wherein the base portion is formed by applying a gel-type natural polymer to the surface or an inside and a surface of the support portion and the frame portion and then freeze-drying it.

12. The artificial support of claim 11, wherein the base portion
surrounds the support portion and the frame portion so that they are not exposed to an outside.
